(19) United States
(12) Patent Application Publication
(10) Pub. No.: US 2024/0315640 A1
(43) Pub. Date: Sep. 26, 2024

(54) TOOL FOR ANALYZING SPEECH USING AI TECHNIQUES TO DIAGNOSE DEMENTIA

(71) Applicant: Drexel University, Philadelphia, PA (US)

(72) Inventors: Hualou Liang, Philadelphia, PA (US); Felix Agbavor, Philadelphia, PA (US)

(21) Appl. No.: 18/613,692

(22) Filed: Mar. 22, 2024

Related U.S. Application Data
(60) Provisional application No. 63/453,892, filed on Mar. 22, 2023.

(51) Int. Cl. A61B 5/00; G06F 40/279; G06N 3/0895

(52) U.S. Cl. CPC A61B 5/4803; A61B 5/4088; A61B 5/7267; A61B 5/7282; G06F 40/279; G06N 3/0895

(57) ABSTRACT

Provided herein are methods of training a machine-learning model to detect a neurological condition. The methods include providing a neurological condition dataset comprising neurological condition text embeddings, the neurological condition text embeddings including large language model (LLM) text embeddings from subjects having the neurological condition; providing a control dataset comprising control text embeddings, the control text embeddings including LLM text embeddings from healthy subjects; and training the machine-learning model using the neurological condition dataset and the control dataset, forming a trained machine-learning model configured to detect a neurological condition. Also provided herein are methods of detecting a neurological condition using the trained machine-learning model.

### CROSS-REFERENCE TO RELATED APPLICATIONS

The present application claims priority under 35 U.S.C. § 119(e) to U.S. Provisional Patent Application No. 63/453,892, filed Mar. 22, 2023, which application is incorporated herein by reference in its entirety.

### BACKGROUND

Alzheimer's disease (AD) is a currently incurable brain disorder. AD is a neurodegenerative disease that involves progressive cognitive declines, including speech and language impairments. It is the most common etiology of dementia, affecting 60-80% cases. Given its prevalence and the lack of a cure for AD, there is an urgent need for early diagnosis of dementia. Early detection is considered paramount in providing the best care to a patient with AD, and would yield clear benefits in improving quality of life for individuals with dementia.

Current diagnoses for AD are still primarily made through clinical assessments such as brain imaging or cognitive tests (e.g., Mini-Mental State Examination (MMSE)) for evaluating the progression of AD. However, current diagnostic processes are often expensive and involve lengthy medical evaluations. Previous studies have shown that spontaneous speech contains valuable clinical information in AD. These prior works on speech analysis are mainly focused on the feature-based approach using acoustic features extracted from the speech audio and the linguistic features derived from the written texts or speech transcripts through NLP techniques. Both the linguistic and acoustic features, sometimes along with other speech characteristics, have been extensively used for dementia classification based on speech data. This feature-based approach, however, relies heavily upon domain specific knowledge and hand-crafted transformations. As a result, it often fails to extract more abstract, high-level representations, and hence is hard to generalize to other progression stages and disease types, which may correspond to different linguistic features.

Therefore, there remains a need in the art for improved methods of early detection of dementia. The present invention addresses this need.

### SUMMARY

In one aspect, a method of training a machine-learning model to detect a neurological condition includes providing a neurological condition dataset comprising neurological condition text embeddings; providing a control dataset comprising control text embeddings; and training the machine-learning model using the neurological condition dataset and the control dataset, forming a trained machine-learning model; wherein the trained machine-learning model is configured to detect a neurological condition. In some embodiments, the neurological condition text embeddings including large language model (LLM) text embeddings from subjects having the neurological condition. In some embodiments, the control text embeddings including LLM text embeddings from healthy subjects. In some embodiments, the text embeddings from subjects having the neurological condition and the text embeddings from healthy subjects capture at least one of lexical, syntactic, and semantic properties. In some embodiments, the neurological condition is dementia or Alzheimer's disease (AD).

In some embodiments, providing the neurological condition dataset comprises generating the neurological condition dataset. In some embodiments, generating the neurological condition dataset includes providing neurological condition audio recordings of speech from the subjects having the neurological condition; converting the neurological condition audio recordings to neurological condition text transcripts; and deriving the neurological condition text embeddings from the neurological condition text transcripts using the LLM. In some embodiments, providing the control dataset comprises generating the control dataset. In some embodiments, generating the control dataset includes providing control audio recordings of speech from the healthy subjects; converting the control audio recordings to control text transcripts; and deriving the control text embeddings from the control text transcripts using the LLM. In some embodiments, converting the neurological condition audio recordings to the neurological condition text transcripts includes inputting the neurological condition audio recordings to an automatic speech recognition model. In some embodiments, converting the control audio recordings to the control text transcripts includes inputting the control audio recordings to an automatic speech recognition model.

In some embodiments, the LLM includes a natural language processing (NLP) LLM. In some embodiments, the LLM is fine-tuned with speech transcripts.

In some embodiments, the neurological condition dataset and the control dataset further comprise at least one acoustic feature. In some embodiments, the at least one acoustic feature includes features related to temporal analysis, frequency analysis, different aspects of speech production, and combinations thereof. In some embodiments, the at least one acoustic feature is combined using concatenation.

In some embodiments, detecting the neurological condition includes distinguishing between subjects having the neurological condition and healthy subjects without the neurological condition. In some embodiments, the machine-learning model includes support vector classifier (SVC), logistic regression (LR), or random forest (RF).

In some embodiments, detecting the neurological condition includes providing a severity prediction for the neurological condition. In some embodiments, the machine-learning model includes a regression model. In some embodiments, the regression model includes support vector regressor (SVR), ridge regression (Ridge), or random forest regressor (RFR).

In another aspect, a method of detecting a neurological condition in a subject includes providing a test dataset from the subject, the test dataset including LLM text embeddings from the subject; and inputting the test dataset to the trained machine-learning model according to any of the embodiments disclosed herein; wherein the trained machine-learning model outputs a detection of the neurological condition based upon the test dataset. In some embodiments, providing the test dataset comprises generating the test dataset. In some embodiments, generating the test dataset includes providing audio recordings of speech from the subject; converting the audio recordings to text transcripts; and deriving the text embeddings from the text transcripts using the LLM. In some embodiments, converting the audio recordings to text transcripts includes inputting the audio recordings to an automatic speech recognition model.

In another aspect, a system for detecting a neurological condition in a subject includes a processor; a memory unit; and a communication interface; wherein the processor is connected to the memory unit and the communication interface; and wherein the processor and memory are configured to implement the method according to any of the embodiments disclosed herein. In some embodiments, the system further includes a web application configured and adapted to provide a user interface for the subject.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1A illustrates a flow diagram of feature data extraction.

FIG. 1B illustrates a flow diagram of text embedding data extraction, in accordance with certain exemplary embodiments of the present disclosure.

FIG. 2 illustrates ROC curves for RF model using the acoustic features and the GPT-3 embeddings.

FIG. 3 illustrates a flow diagram of data extraction and use in connection with a machine-learning model, in accordance with certain exemplary embodiments of the present disclosure.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

Provided herein are artificial intelligence (AI)-driven methods of detecting neurological conditions. In some embodiments, the method includes training a machine-learning model to detect a neurological condition in a subject. In some embodiments, the method includes training the machine-learning model using embeddings from a large language model (LLM). For example, in some embodiments, the method includes providing a neurological condition dataset comprising neurological condition text embeddings; providing a control dataset comprising control text embeddings; and training the machine-learning model using the neurological condition dataset and the control dataset. The neurological condition text embeddings and the control text embeddings include large language model (LLM) text embeddings from subjects having the neurological condition and from healthy subjects, respectively. The healthy subjects include any subject without the neurological condition, including subjects without a specific neurological condition of interest and/or subjects without any neurological condition. The training of the machine-learning model forms a trained machine-learning model configured to detect a neurological condition.

In some embodiments, providing the neurological condition dataset and/or the control dataset includes generating the neurological condition dataset and/or control dataset, respectively. In some embodiments, the text embeddings capture one or more linguistic features, such as, but not limited to, lexical, syntactic, and/or semantic properties. In some embodiments, generating the neurological condition dataset includes providing neurological condition audio recordings of speech from the subjects having the neurological condition; converting the neurological condition audio recordings to neurological condition text transcripts; and deriving the neurological condition text embeddings from the neurological condition text transcripts using the LLM. Similarly, in some embodiments, generating the control dataset includes providing control audio recordings of speech from the healthy subjects; converting the control audio recordings to control text transcripts; and deriving the control text embeddings from the control text transcripts using the LLM.

In some embodiments, converting the neurological condition and/or control audio recordings to the neurological condition and/or control text transcripts includes inputting the respective audio recordings to an automatic speech recognition model. The audio recordings may be converted to text transcripts using any suitable automatic speech recognition model, such as, but not limited to, Wav2Vec 2.0., Hidden Markov Models, and Neural Networks. In some embodiments, for example, converting the audio recordings to text transcripts includes loading each audio file as a waveform, tokenizing the waveform, feeding the tokenized waveform into the model for speech recognition, and decoding the tokenized waveform as text transcripts.

The LLM includes any LLM suitable for generating the text embeddings disclosed herein. In some embodiments, the LLM includes a natural language processing (NLP) LLM. For example, one suitable LLM includes the generative pre-trained transformer 3 (GPT-3) available from Open AI. Other LLMs include OpenAI's GPT-4, Anthropic's Claude, and Google DeepMind's Gemini. Although discussed herein primarily with respect to GPT-3, as will be appreciated by those skilled in the art, the disclosure is not so limited and may include any other suitable LLM. In some embodiments, the LLM is fine-tuned with speech transcripts.

In some embodiments, the neurological condition dataset and the control dataset further comprise at least one acoustic feature. The at least one acoustic feature may be combined with the respective text embedding in any suitable manner, such as, but not limited to, through concatenation. Suitable acoustic features include, but are not limited to, features related to temporal analysis (e.g., pause rate, phonation rate, periodicity of speech, etc.), frequency analysis (e.g., mean, variance, kurtosis of Mel frequency cepstral coefficients), different aspects of speech production (e.g., prosody, articulation, or vocal quality), and/or combinations thereof. For example, in some embodiments, the acoustic features include at least one of a low-level descriptor (e.g., pitch, jitter, formant 1-3 frequency and relative energy, shimmer, loudness, alpha ratio, Hammarberg index, etc.), a functional applied to pitch and loudness, a statistic over the unvoiced segments, a temporal feature, additional cepstral parameters and dynamic parameters, and combinations thereof. In some embodiments, the acoustic features are extracted directly from speech through any suitable manner. For example, in one embodiment, the acoustic features are extracted using open-source Speech and Music Interpretation by Large-space Extraction (OpenSMILE).

In some embodiments, detecting the neurological condition includes distinguishing between subjects having the neurological condition and healthy subjects without the neurological condition. In some such embodiments, the machine-learning model includes any suitable classification model, such as, but not limited to, support vector classifier (SVC), logistic regression (LR), and/or random forest (RF). Additionally or alternatively, in some embodiments, detecting the neurological condition includes providing a severity prediction for the neurological condition. In some such embodiments, the machine-learning model includes any suitable regression model, such as, but not limited to, support vector regressor (SVR), ridge regression (Ridge), or random forest regressor (RFR). In some embodiments, predicting the severity of the neurological condition includes using a subject's Mini-Mental State Examination (MMSE) score.

Speech, including spontaneous speech, can serve as an important biomarker of various neurological conditions, any of which can be detected using the machine-learning model trained according to the embodiments disclosed herein. In some embodiments, using speech in connection with the machine-learning model described herein provides quick, cheap, accurate, and non-invasive diagnosis of neurological conditions. Additionally or alternatively, in some embodiments, the machine-learning model described herein may be used to provide early screening for neurological conditions. Suitable neurological conditions include, but are not limited to, neurodegenerative disorders (e.g., dementia, Alzheimer's disease (AD), Huntington's disease, etc.), movement disorders (e.g., Parkinson's disease, etc), or any other suitable neurological condition. For example, in some embodiments, the neurological condition includes AD.

Accordingly, also provided herein are methods of detecting a neurological condition in a subject. In some embodiments, the method includes predicting and/or assessing the status of a neurological condition in a subject by analyzing speech using embeddings from a LLM. In some embodiments, the method includes providing a test dataset from the subject, the test dataset including LLM text embeddings from the subject; and inputting the test dataset to the machine-learning model trained according to any of the embodiments disclosed herein. After inputting the test dataset, the trained machine-learning model outputs a detection of the neurological condition based thereon. In some embodiments, providing the test dataset includes generating the test dataset. For example, generating the test dataset may include providing audio recordings of speech from the subject; converting the audio recordings to text transcripts; and deriving the text embeddings from the text transcripts using the LLM. In some embodiments, converting of the audio recordings to text transcripts includes inputting the audio recordings to an automatic speech recognition model.

Further provided herein are systems for detecting a neurological condition in a subject. In some embodiments, the system includes a processor, a memory unit, and a communication interface. The processor is connected to the memory unit and the communication interface, and the processor and memory are configured to implement the method according to any of the embodiments disclosed herein. For example, in some embodiments, the system is configures to detect a neurological condition in a subject according to any of the embodiments disclosed herein. In some embodiments, the system includes a web application configured and adapted to provide a user interface for the subject. Additionally or alternatively, in some embodiments, the system includes a personal computing device and/or a microphone used to record audio. In some embodiments, the system is configured to communicate with an LLM to generate the text embeddings described herein.

Still further provided herein is a computer readable storage medium storing computer-executable instructions for performing the method according to any of the embodiments disclosed herein.

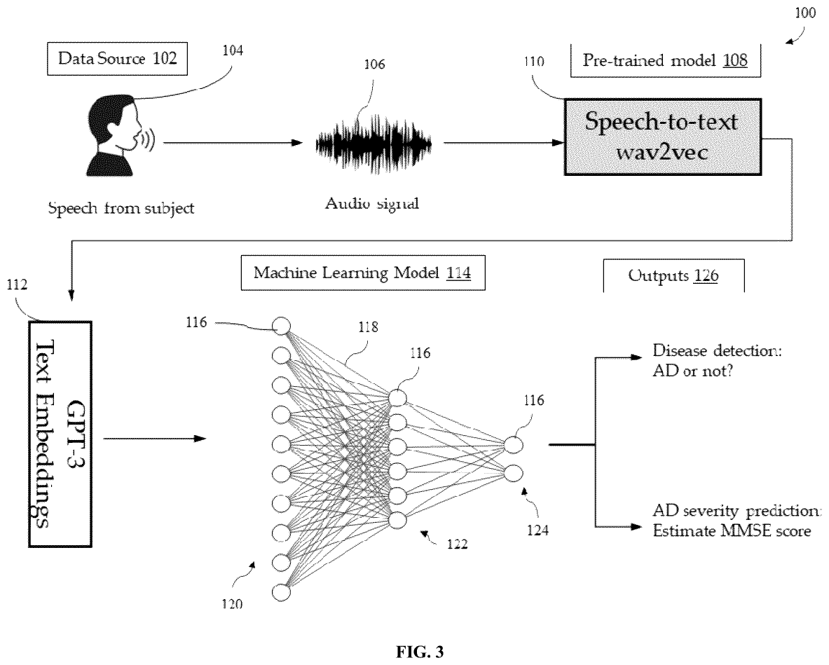

Referring now to FIG. 3, an exemplary flow diagram and method of detect neurological conditions using text embeddings from LLMs is shown. As illustrated therein, in some embodiments, the method includes obtaining an audio signal 106 from a data source 102 (e.g., speech from a test subject or patient 104), converting the audio signal 106 to a text transcript (e.g., using speech-to-text module 110 like “wav2vec”), inputting the text transcript to a pre-trained model 108 that generates text embeddings 112 (e.g., an LLM such as GPT-3), inputting the text embeddings 112 to a machine-learning model 114 (e.g., the machine-learning model trained according to any of the embodiments disclosed herein), and obtaining an output 126 from the machine-learning model 114, the output providing detection information for the neurological condition. As described elsewhere herein, the audio signal 106 can be provided (e.g., pre-recorded) or can be obtained using any suitable audio recording device. As also described elsewhere herein, the text embeddings can include vector representations of the data set, the vector representations including linguistic features.

Still referring to FIG. 3, the machine-learning model 114 is illustrated as including a plurality of nodes 116 arranged in a multilayer neural network. Each node is connected to at least one other node by an association 118. In some embodiments, the leftmost column of nodes is considered to include input nodes 120, the middle column of nodes is considered to include a plurality of intermediate layers 122 (or hidden layers, where only one layer is illustrated for simplicity), and the rightmost column of nodes is considered to include output nodes 124. In such embodiments, the output nodes 124 generate the outputs 126 regarding a neurological condition (or lack thereof). As illustrated, the outputs 126 can include detection of a neurological condition in the subject 104 and/or a prediction regarding the severity of a neurological condition (e.g., by providing an estimated MMSE score).

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### Examples

Large language models (LLMs), which have demonstrated impressive performance on many natural language processing (NLP) tasks, provide powerful universal language understanding and generation. For example, GPT-3, or Generative Pre-trained Transformer 3 (produced by OpenAI) is one of the largest existing language models. There are four GPT-3 models available to the public via the OpenAI API, each having different number of embedding size and parameter: Ada (1024 dimensions, 300M), Babbage (2048 dimensions, 1.2B), Curie (4096 dimensions, 6B), and Davinci (12288 dimensions, 175B). Of these models, Ada is the fastest and most affordable, but also the least capable, while Davinci is the most powerful and most expensive.

GPT-3 has been shown to be particularly effective in zero-shot learning (i.e., zero-data learning), where the language model is adapted to downstream tasks, such as translation, text summarization, question-answering and dialogue systems, without the need for additional, task-specific data. Additionally, GPT-3 has been shown to be particularly effective in encoding a wealth of semantic knowledge about the world and producing a learned representation (embedding), typically a fixed-size vector, that lends itself well to discriminative tasks. Each dimension of an embedding (e.g., text embedding), can be referred to as a feature. A feature represents a certain universal characteristic about text according to how the model understands it. The text embeddings entail meaningful vector representations that can uncover additional patterns and characteristics, as captured in the semantic meaning of the input, that might not be evident even to trained experts.

With the understanding that language impairment is an important biomarker of neurodegenerative disorders such as Alzheimer's disease (AD), this Example explores whether LLM's (e.g., GPT-3 from OpenAI) can be used for early prediction of dementia through speech. More specifically, this Example studies the extent to which text embeddings generated by GPT-3 can be utilized to predict neurological conditions (e.g., dementia, AD, etc.). Data from the Alzheimer's Dementia Recognition through Spontaneous Speech only (ADReSSo) Challenge was used as a shared task for the systematic comparison of approaches to the detection of cognitive impairment and decline based on spontaneous speech. With this dataset, two tasks were performed: (1) an AD classification task for distinguishing individuals with AD from healthy controls, and (2) an MMSE score regression task to infer the cognitive test score of the subject, both solely based on the demographically matched spontaneous speech data.

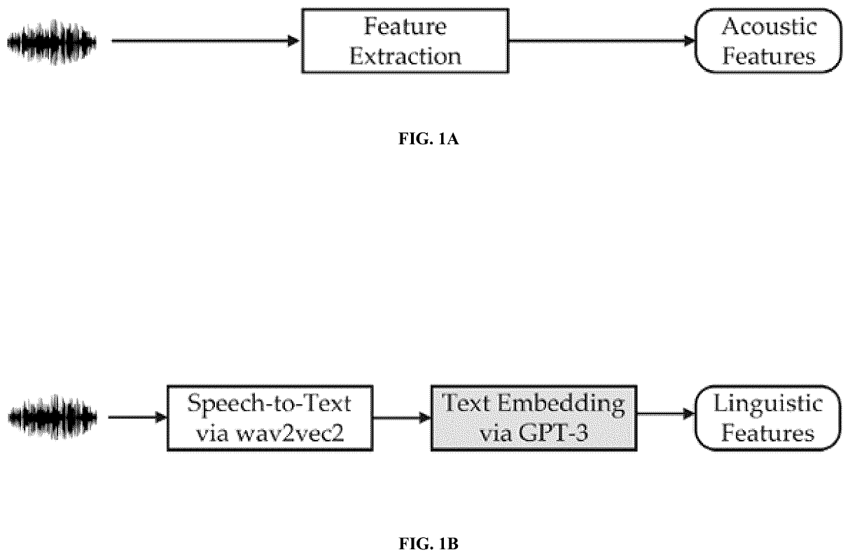

The vast semantic knowledge encoded in the GPT-3 model can be leveraged to generate text embedding, a vector representation of the transcribed text from speech, that captures the semantic meaning of the input. It is shown herein that text embeddings can be reliably used for detection of Alzheimer's dementia and inference of the cognitive testing score, both solely based on speech data. It is also illustrated that text embedding (e.g., see FIG. 1B) considerably outperforms the conventional acoustic feature-based approach (e.g., see FIG. 1A) and is even competitive with fine-tuned models. Taken together, the results of this Example demonstrate that LLM based text embedding, derived from GPT-3 model, can be utilized for the assessment of AD status and/or prediction of dementia directly from spontaneous speech, which facilitates improved early diagnosis of dementia. These results further illustrate the feasibility of fully deployable AI-driven tools for early diagnosis of dementia and direct tailored interventions to individual needs, thereby improving quality of life for individuals with dementia.

### Results

The results from two tasks (which include AD vs non-AD classification and AD severity prediction using a subject's MMSE score) are provided herein. For the classification task, either the acoustic features or GPT-3 embeddings (Ada and Babbage) or both are fed into a machine-learning model such as support vector classifier (SVC), logistic regression (LR) or random forest (RF).

As a comparison, finetuning on the GPT-3 model was further performed to see if there is any advantage over the GPT-3 embedding. For the AD severity prediction, a regression analysis was performed based on both the acoustic features and GPT-3 embeddings to estimate a subject's MMSE score using three regression models, i.e., support vector regressor (SVR), ridge regression (Ridge) and random forest regressor (RFR).

### AD vs Non-AD Classification

Provided herein are the AD classification results between AD and non-AD (or healthy control) subjects based on different features: GPT-3 based text embeddings, the acoustic features, and their combination. The GPT-3 based text embeddings were benchmarked against the mainstream fine-tuning approach. It was shown that the GPT-3 based text embeddings considerably outperform both the acoustic feature-based approach and the finetuned model.

### Using Acoustic Features

The classification performance in terms of accuracy, precision, recall and F1 score for all the models with the acoustic features is shown in Table 1 for both the 10-fold cross-validation (CV) and evaluation on the held-out test set not used in any way during model development. F1 score is an alternative machine-learning evaluation metric that assesses the predictive skill of a model. This assessment is done by elaborating on its class-wise performance rather than an overall performance (e.g., as done by accuracy). F1 scores can combine two competing metrics—precision and recall scores of a model. Table 1 illustrates model performances obtained by the 10-fold CV (top) where the mean (standard deviation) are reported, and evaluated on test set (bottom) for AD classification using acoustic features. The bolded data indicate the best overall performance for the metric.

TABLE 1
Model performance obtained by the 10-fold CV (top) where the
mean (standard deviation) are reported, and evaluated on test
set (bottom) for AD classification using acoustic features.

| | Model | Accuracy | Precision | Recall | F1 |
|---|---|---|---|---|---|
| 10- | SVC | 0.697 | 0.722 | 0.660 | 0.678 |
| fold | | (0.095) | (0.091) | (0.120) | (0.084) |
| CV | LR | 0.632 | 0.645 | 0.656 | 0.647 |
| | | (0.120) | (0.136) | (0.131) | (0.121) |
| | RF | 0.668 | 0.705 | 0.704 | 0.686 |
| | | (0.101) | (0.156) | (0.114) | (0.084) |
| Test | SVC | 0.634 | 0.657 | 0.622 | 0.639 |
| Set | LR | 0.620 | 0.600 | 0.618 | 0.609 |
| | RF | 0.746 | 0.771 | 0.730 | 0.750 |

Table 1 indicates that, for the evaluation on the test set, RF performs the best among the three models in all the metrics used. For the 10-fold CV, RF also has the highest recall and F1 score among all the models, although the SVC performs better than other two models in both accuracy and precision.

### Using GPT-3 Embeddings

The classification performance of the GPT-3 embedding models is shown in Table 2 for the 10-fold CV and for the evaluation on the test set. As illustrated in Table 2, the use of GPT-3 embeddings yields a substantial improvement in performance when compared to the acoustic feature-based approach (Table 1). Additionally, Table 2 illustrates that Babbage outperforms Ada, a result consistent with the general notion that larger model is more powerful in various tasks. Table 2 also illustrates the performance of the 10-fold CV is comparable to that for the evaluation on the test set on the hold-out test set. Table 2 further illustrates that a direct comparison with the best baseline of the classification accuracy of 0.6479 on the same test set reveals that GPT-3 performs remarkably well with the best accuracy of 0.8028 by SVC, showing clear advantage of using GPT-3 models.

TABLE 2
Model performance obtained by the 10-fold CV (top) where the mean (standard
deviation) are reported, and evaluated on test set (bottom) for AD classification
using text embeddings from the GPT-3 base models (Babbage and Ada).

| | Embeddings | Model | Accuracy | Precision | Recall | F1 |
|---|---|---|---|---|---|---|
| 10-fold CV | Ada | SVC | 0.788 (0.075) | 0.798 (0.109) | 0.819 (0.098) | 0.799 (0.066) |
| | | LR | 0.796 (0.107) | 0.798 (0.126) | 0.835 (0.129) | 0.808 (0.100) |
| | | RF | 0.734 (0.090) | 0.738 (0.109) | 0.763 (0.149) | 0.743 (0.103) |
| | Babbage | SVC | 0.802 (0.054) | 0.823 (0.092) | 0.804 (0.103) | 0.806 (0.053) |
| | | LR | 0.809 (0.112) | 0.843 (0.148) | 0.811 (0.091) | 0.818 (0.091) |
| | | RF | 0.760 (0.052) | 0.780 (0.102) | 0.781 (0.110) | 0.770 (0.047) |
| Test Set | Ada | SVC | 0.788 | 0.708 | 0.971 | 0.819 |
| | | LR | 0.718 | 0.653 | 0.914 | 0.762 |
| | | RF | 0.732 | 0.690 | 0.829 | 0.753 |
| | Babbage | SVC | 0.803 | 0.723 | 0.971 | 0.829 |
| | | LR | 0.718 | 0.647 | 0.943 | 0.767 |
| | | RF | 0.761 | 0.714 | 0.857 | 0.779 |

To examine how the GPT-3 based text embeddings fare with the fine-tuning approach, GPT-3 Babbage was used as the pretrained model and fine-tuned with speech transcripts. The results shown in Table 3 are for both the 10-fold CV and evaluation on the test set. It can be seen in Table 3 that, while the overall performance is comparable for both the 10-fold CV and the evaluation on the test set, the fine-tuned Babbage model underperforms the GPT-3 based text embeddings.

TABLE 3
Results for the fine-tuned GPT-3 Babbage model obtained by
the 10-fold CV where the mean (standard deviation) are reported,
and evaluated on test set for AD classification.

| | Accuracy | Precision | Recall | F1 |
|---|---|---|---|---|
| 10- | 0.797 (0.058) | 0.810 (0.127) | 0.809 (0.071) | 0.797 (0.105) |
| fold | | | | |
| CV | | | | |
| Test | 0.803 | 0.806 | 0.806 | 0.806 |
| Set | | | | |

### Combination of Acoustic Features and GPT-3 Embeddings

To evaluate whether the acoustic features and the text embeddings can provide complementary information to augment the AD classification, the acoustic features from speech audio data were combined with the GPT-3 based text embeddings by simply concatenating them. Table 4 shows the results for both the 10-fold CV and evaluation on the test set for different machine-learning models. With additional acoustic features, only marginal improvements in the classification performance can be observed on the 10-fold CV. There is no clear difference in predicting the test set in terms of accuracy and F1 score when the acoustic features are combined with GPT-3 based text embeddings, but we instead observe higher precision at the expense of lower recall. This observation indicates that the combined approach could be well-suited in screening AD when high precision is much more important than the recall.

TABLE 4
Model performance for the 10-fold CV with standard deviation
and the evaluation on test set using a combination of the
GPT-3 Babbage embeddings and the acoustic features.

| | Model | Accuracy | Precision | Recall | F1 |
|---|---|---|---|---|---|
| 10- | SVC | 0.814 | 0.838 | 0.802 | 0.814 |
| fold | | (0.115) | (0.133) | (0.136) | (0.119) |
| CV | LR | 0.800 | 0.831 | 0.803 | 0.809 |
| | | (0.108) | (0.137) | (0.097) | (0.093) |
| | RF | 0.731 | 0.741 | 0.762 | 0.745 |
| | | (0.121) | (0.141) | (0.119) | (0.109) |
| Test | SVC | 0.802 | 0.971 | 0.723 | 0.829 |
| Set | LR | 0.676 | 0.971 | 0.607 | 0.747 |
| | RF | 0.788 | 0.914 | 0.727 | 0.810 |

Comparison of Acoustic Features with GPT-3 Embeddings

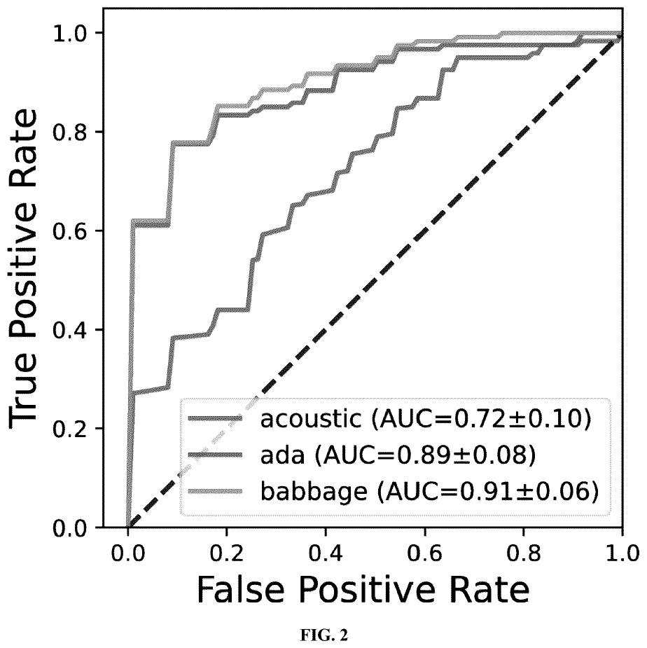

To compare the acoustic features with the GPT-3 embeddings, further analysis was performed based on the performance measurement of the area under the Receiver Operating Characteristic (ROC) curve (AUC). FIG. 2 shows the ROC curves for RF model using the acoustic features (the best-performing acoustic model) and the GPT-3 embeddings (both Ada and Babbage).

The mean and standard deviation of AUCs from the 10-fold CV are also reported, which indicate that the GPT-3 embeddings outperform the RF model using the acoustic features and Babbage is marginally better than Ada. The Kruskal-Wallis H-test reveals a significant difference between the GPT-3 embeddings and the RF acoustic model (H=5.622, p<0.05).

Comparison with Several Existing Models

The GPT-3 embedding (Babbage) method of the present Example was benchmarked against other state-of-the-art AD detection models. The existing methods include the studies from Luz et al., Balagopalan & Novikova, and Pan et al., which all used the ADReSSo Challenge data. The models selected are all trained based on the 10-fold CV and evaluated on the same unseen test set to ensure fair comparison. For example, Model 4 & 5 are not included in Pan et al. as the models were trained by holding out 20% of the training set. Instead, the best model (Model 2) was selected, which was trained using 10-fold CV. The comparison is presented in Table 5, from which we can see that our method overall outperforms all other models in terms of accuracy, recall, and F1 score, though the precision is relatively low.

TABLE 5
Performance comparison between our model and other
models on the ADReSSo 2021 unseen test set.

| | Model | Accuracy | Precision | Recall | F1 |
|---|---|---|---|---|---|
| GPT-3 | SVC | 0.803 | 0.723 | 0.971 | 0.829 |
| Embedding | | | | | |
| Pan et | BERTbase | 0.803 | 0.862 | 0.714 | 0.781 |
| al. 2021 | | | | | |
| Balagopalan | SVC | 0.676 | 0.636 | 0.800 | 0.709 |
| et al 2021 | | | | | |
| Luz et | SVC | 0.789 | 0.778 | 0.800 | 0.789 |
| al. 2021 | | | | | |

### MMSE Score Prediction

A regression analysis was performed using three different models: Support Vector Regression (SVR), Ridge Regression (Ridge) and Random Forest Regressor (RFR). The regression results, reported as root mean squared error (RMSE), using acoustic features and text embeddings from GPT-3 (Ada and Babbage) are shown in Tables 6 and 7, respectively. In each table, the RMSE scores of the MMSE prediction are provided for both the 10-fold CV and evaluation on the test set, with the best RMSE score being bold.

TABLE 6
MMSE prediction in terms of RMSE scores for three
different models (SVR, Ridge and RFR) using acoustic
features on the 10-fold CV (top) with standard deviation
and on the inference on test set (bottom).

| | Model | RMSE |
|---|---|---|
| 10- | SVR | 7.049 (2.355) |
| fold | Ridge | 6.768 (1.524) |
| CV | RFR | 6.901 (1.534) |
| Test | SVR | 6.285 |
| Set | Ridge | 6.250 |
| | RFR | 6.434 |

TABLE 7
MMSE prediction in terms of RMSE scores for three different
models (SVR, Ridge and RFR) using text embeddings from
GPT-3 (Ada and Babbage) on the 10-fold CV (top) with standard
deviation and on the inference on test set (bottom).

| | Embeddings | Model | RMSE |
|---|---|---|---|
| 10- | Ada | SVR | 6.097 (2.057) |
| fold | | Ridge | 6.058 (1.298) |
| CV | | RFR | 6.300 (1.129) |
| | Babbage | SVR | 5.976 (1.173) |
| | | Ridge | 5.843 (1.037) |
| | | RFR | 6.330 (1.032) |
| Test | Ada | SVR | 5.6307 |
| Set | | Ridge | 5.8735 |
| | | RFR | 6.0010 |
| | Babbage | SVR | 5.4999 |
| | | Ridge | 5.4645 |
| | | RFR | 5.8142 |

With acoustic features, Table 6 illustrates that Ridge has the lowest RMSE score (6.2498) for MMSE prediction on the evaluation on the test set and the lowest RMSE of 6.7683 on the 10-fold CV. With the GPT-3 based text embeddings, Table 7 illustrates that Babbage has better prediction performance than Ada in terms of RMSE score in both the 10-fold CV and evaluation on the test set. When comparing the overall regression results in relation to what kinds of features are used, the GPT-3 based text embeddings provide clear advantage, as they always outperform the acoustic features.

### Discussion

GPT 3, a specific large language model produced by OpenAI, is particularly powerful in encoding a wealth of semantic knowledge about the world and producing a high-quality vector representation (embedding) that lends itself well to discriminative tasks. GPT-3, and other LLM's useful in NLP, can be used to predict dementia from speech by utilizing the vast semantic knowledge encoded in the model. Results presented herein demonstrate that text embedding, generated by GPT-3, can be reliably used to detect individuals with AD from healthy controls and also to infer the subject's cognitive testing score, both solely based on speech data. Text embedding outperforms the conventional acoustic feature-based approach and even performs competitively with fine-tuned models. These results suggest that GPT-3 based text embedding can be used for AD assessment and has the potential to improve early diagnosis of dementia. Studies provided herein performed model development and internal validation based mainly on ADReSSo Challenge data.

When deciding which model should be used (e.g., Ada, Babbage, Curie, Davinci, etc.), the embedding size and the number of parameters are two important factors that should be taken into consideration. In general, larger models incur higher cost in terms of storage, memory, and computation time, which inevitably has direct impact on the model deployment in real-world applications (e.g., in AD diagnosis). Given the budget consideration and especially small data sample in the ADReSSo Challenge, Ada and Babbage models were studied herein. There can be a risk of overfitting when the data are not abundant, especially with the larger models (e.g., Curie and Davinci). Indeed, when tested with the Curie and Davinci, the model was found to be overfitting by observing almost perfect recall and extremely low precision in AD classification task. It should be noted that, while large sample sizes can certainly help in certain applications, the studies provided herein have taken precautious steps to test model generalizability with both the 10-fold CV and evaluation on the test set to guard against the problem of small sample size.

Fine-tuning has become the de facto standard to leverage large pretrained models to perform downstream tasks. When GPT-3 Babbage was used as the pretrained model and fine-tuned it with the speech transcripts, an improvement in performance was not seen, as could be expected. The results presented herein illustrate that GPT-3 embedding model performs competitively with fine-tuned models; however, there is a possibility that the underperformance could be due to the insufficient data available in this task, as it is well known that the fine-tuning may predispose the pretrained model to overfitting due to the huge model and relatively small size of the domain-specific data.

A fully deployable AI-driven speech analysis for early diagnosis of dementia and direct tailored interventions to individual needs can be developed and translated. However, major challenges lie with data quality (e.g., inconsistency and instability), data quantity (e.g., limited data), and diversity. For any models to work well, a very large, diverse and robust set of data is needed. Leveraging AI with the growing development of largescale, multi-modal data (e.g., neuroimaging, speech and language, behavioral biomarkers, patient information on electronic medical records, etc.) can help alleviate the data problem and allow for more accurate, efficient, and early diagnosis.

Certain embodiments of the present disclosure (including an AI model) can be deployed as a web application or a voice-powered app used at the doctor's office to aid clinicians in AD screening and early diagnosis. When applying AI and machine-learning to predict dementia in clinical settings, there are however a number of considerations. First, the bias should be considered in model development. Speech data from around the world, in many different languages, can be included to guard against this problem, and to ensure the models work can work for all (or nearly all) patients regardless of certain factors (e.g., age, gender, ethnicity, nationality and other demographic criteria). It is preferred to develop ethical and legal systems for the implementation, validation and control of AI in clinical care. Second, the privacy is a major concern in this nascent field, particularly speech data, which can be used to identify individuals. Third, there is a need to establish trust in AI, especially pertinent to the so-called ‘black box’ problem. This often arises in machine-learning models where even the developers themselves can't fully explain, particularly which information are used to make predictions. This can be problematic in clinical practice to explain how a diagnosis of dementia is ascertained and what can determine personalized treatments. Explainable AI aims to address the questions about the decision-making processes. AI can provide augmented decision making in driving efficient care and helping make accurate diagnoses. Before the AI-driven technologies enter mainstream use in aiding the diagnosis of dementia, rigorous validation from large-scale, well-designed representative studies through multidisciplinary collaboration between AI researchers and clinicians can be provided. Methods provided herein (implementing AI) can improve early diagnosis of neurological conditions; early diagnosis is important to improve quality of life for individuals with dementia.

### Materials and Methods

### Dataset Description

The dataset used in this study is derived from the ADReSSo Challenge [21], which consists of set of speech recordings of picture descriptions produced by cognitively normal subjects and patients with an AD diagnosis, who were asked to describe the Cookie Theft picture from the Boston Diagnostic Aphasia Examination. There are totally 237 speech recordings, with 70/30 split balanced for demographics, resulting in 166 and 71 in the training set and the test set, respectively. In the training set, there are 87 samples from AD subjects and 79 from non-AD (or healthy control) subjects. The datasets were matched so as to avoid potential biases often overlooked in assessment of AD detection methods, including incidences of repetitive speech from the same individual, variations in speech quality, and imbalanced distribution of gender and age. The detailed procedures to match the data demographically according to propensity scores were described in Luz et al. In the final dataset, all standardized mean differences for the age and gender covariates are <0.001.

### Computational Approaches

Text embedding (e.g., see FIG. 1B) from GPT-3, which can be readily accessed via OpenAI Application Programming Interface (API), is provided in certain exemplary embodiments of the present disclosure. The OpenAI API, powered by a family of models with different capabilities and price points, can be applied to virtually any task that involves understanding or generating natural language or code. GPT-3 can be used for text embedding, which is powerful representation of the semantic meaning of a piece of text. In results of the studies provided herein, a GPT-3 embedding approach is benchmarked against both the conventional acoustic feature-based approach (FIG. 1A) and the prevailing fine-tuned model.

Text Embeddings from GPT-3

An innovative use of text embeddings to predicting dementia from speech, powered by GPT-3, is provided in certain exemplary embodiments provided herein. In an exemplary method provided herein (e.g., illustrated at least in part in FIG. 1B), voice (e.g., files, live recordings, etc.) can be converted to text using a pretrained model for automatic speech recognition (e.g., Wav2Vec 2.0, a state-of-the-art model). In certain studies provided herein, the base model wav2vec2--base-960h was used; such a model was pretrained and fine-tuned on 960 hours of Librispeech on 16 kHz sampled speech audio, which can be accessed from Huggingface. Each audio file can be loaded as a waveform (e.g., with librosa, a python package dedicated to analyzing sounds). The waveform can be tokenized (e.g., using Wav2Vec2Tokenizer) and if necessary, divided them into smaller chunks (e.g., with the maximum size of 100,000, as in the studies provided herein) to fit into memory, which can subsequently be fed into a wav2vec model for speech recognition (e.g., Wav2Vec2ForCTC) and decoded as text transcripts.

GPT-3 based text embeddings can then be derived from the transcribed text obtained via wav2vec2. In studies provided herein, the endpoint in the OpenAI API was used to access GPT-3 embedding models. These embeddings entail meaningful vector representations that can capture lexical, syntactic, and semantic properties useful for dementia classification. GPT-3 based text embeddings results can be achieved in a variety of tasks including semantic search, clustering, and classification.

There are four GPT-3 models on a spectrum of embedding size: Ada (1024 dimensions), Babbage (2048 dimensions), Curie (4096 dimensions) and Davinci (12288 dimensions). Davinci is the most powerful but is more expensive than the other models, whereas Ada is the least capable but is significantly faster and cheaper. These embeddings are can be used as features to train machine-learning models for AD assessment. The results obtained by the Ada and Babbage models are provided herein.

Acoustic Feature Extraction from Speech

Conventional acoustic feature-based approach (FIG. 1A) will be used as benchmark for comparison. The acoustic features considered are mainly related to temporal analysis (e.g. pause rate, phonation rate, periodicity of speech, etc.), frequency analysis (e.g. mean, variance, kurtosis of Mel frequency cepstral coefficients) and different aspects of speech production (e.g. prosody, articulation, or vocal quality). In the studies provided herein, acoustic features were extracted directly from speech using open-source Speech and Music Interpretation by Large-space Extraction (OpenSMILE), a widely used open-source toolkit for audio feature extraction and classification of speech and music signals. The studies provided herein primarily used the extended Geneva Minimalistic Acoustic Parameter Set (eGeMAPS) features due to their potential to detect physiological changes in voice production, as well as theoretical significance and proven usefulness in previous studies. There are, in total, 88 features: the arithmetic mean and coefficient of variation of 18 low-level descriptors (e.g., pitch, jitter, formant 1-3 frequency and relative energy, shimmer, loudness, alpha ratio and Hammarberg index etc), 8 functionals applied to pitch and loudness, 4 statistics over the unvoiced segments, 6 temporal features, and 26 additional cepstral parameters and dynamic parameters. This feature set once obtained can be used directly as inputs to a machine-learning model.

Fine-Tuning with Speech Transcripts

Fine-tuning is the prevalent paradigm for using LLMs to perform downstream tasks. In this approach, the pretrained models such as the BERT (Bidirectional Encoder Representations from Transformers), either some or all the model parameters, can be finetuned or updated with downstream task-specific data. Recent work has shown encouraging results with fine-tuned BERT for AD detection. In the studies provided herein, methods employing GPT-based embedding are benchmarked against the mainstream use of fine-tuned model. As such, GPT-3 was used as the pretrained model and was fine-tuned with speech transcripts obtained by wav2vec2 from raw audio files.

To fine tune the custom GPT-3 models, the OpenAI command-line interface was used. Instructions about fine-tuning can be provided to prepare training data (e.g., consisting of 166 paragraphs, totaling 19,123 words that can be used to fine tune one of the base models, such as Babbage and Ada) with speech transcripts.

### Experimental Tasks

AD vs Non-AD Classification. An AD classification task consists of creating a binary classification model to distinguish between AD and non-AD speech. The model can use acoustic features from speech, linguistic features (e.g., embeddings) from transcribed speech, or both. In the studies provided herein used (and methods provided herein can use): (1) the acoustic features extracted from speech audio data, (2) the text embeddings from each GPT-3 base model (Babbage or Ada), and (3) the combination of both as inputs for three different kinds of commonly used machine-learning models, including Support Vector Classifier (SVC), Random Forest (RF), and Logistic Regression (LR). The studies provided herein used the scikit-learn library for the implementation of these models. The hyperparameters for each model are tuned using the 10-fold cross-validation. Specifically, there are two key parameters (the regularization parameter and the kernel coefficient) for SVC trained with a radial basis function kernel, the L2-penalty parameter for LR and two key parameters (the number of estimators and the maximum depth of the tree) for RF. As a comparison, the studies provided herein also fine tune the GPT-3 model (Babbage) with the speech transcripts to assess if the GPT-3 based text embeddings can be better used to predict the dementia.

MMSE Score Prediction. MMSE is perhaps the most common measure for assessing the severity of AD. The studies provided herein performed regression analysis using both the acoustic features and text embeddings from GPT-3 (Ada and Babbage) to predict the MMSE score. The scores normally range from 0 to 30, with scores of 26 or higher being considered normal. A score of 20 to 24 suggests mild dementia, 13 to 20 suggests moderate dementia, and less than 12 indicates severe dementia. As such, the prediction can be clipped to a range between 0 and 30. Three kinds of regression models are employed, including Support Vector Regression (SVR), Ridge regression (Ridge) and Random Forest Regressor (RFR). The models are similarly implemented with the scikit-learn library, with the hyperparameters for each model determined using gridsearch 10-fold cross-validation on the training dataset.

### Performance Evaluation

For AD classification task, the performance can be evaluated by a panel of metrics such as the accuracy, precision, recall and F1-score, where the threshold of 0.5 can be used. The ADReSSo Challenge dataset was split into the training set and the test set, with 70% of samples allocated to the former and 30% allocated to the latter. To evaluate the generalization ability of the model, two ways of reporting the performance are (1) 10-fold cross-validation (CV) and (2) evaluation on the test set. The model was well calibrated before testing. Using the 10-fold CV approach, all the available data (i.e., the entire data including the training set and test set) can be partitioned into three sets (i.e., training, validation and test sets) in an 80/10/10 ratio using the 10-fold CV. That is, 8-fold was used for training, 1-fold was used for validation, and the remaining was used for testing in each run. The average of the ten independent runs was reported in which the test data is different in each run. As such, the potential sampling bias can be reduced where the results can depend on a particular random choice of the data sets. The averaged AUC scores were reported, along with the corresponding standard deviations over the 10-fold CV when comparing the different models using acoustic features, GPT-3 embeddings (both Ada and Babbage) for AD classification.

The second way to report the performance can be to use and evaluate the model on an unseen test set not used in any way during model development. In the studies provided herein, a separate test set that was set aside was used as the independent, held-out dataset. The 10-fold cross-validation was performed but only the existing training set. That is, the training dataset was split into ten folds, with 9 folds for training and the remaining for validation to tune hyperparameters in each run for ten independent runs. The model was fit on the training dataset using the hyperparameters of the best model; the final model was then used on the held-out test data. The use of the held-out test data allows us to directly compare the different models as well as with Challenge baseline on the same dataset. The test dataset was different between 10-fold CV and evaluation on the test set.

For an AD regression task, in the studies provided herein, the 10-fold CV and inference were conducted on the test set. The root mean squared error (RMSE) for the MMSE score predictions on the testing data (using the models obtained by 10-fold CV) were reported. The hyperparameters for each model were determined based on performance in grid-search 10-fold cross-validation on the training dataset.

In finetuning GPT-3 for the AD classification task, the hyperparameters used and tuned include the number of epochs, batch size and learning rate multiplier. The number of epochs were varied from 1 to 5, learning rate multiplier between 0.02 to 0.2 and the batch size between 4 to 10 and compare with the results from default internal parameters originally set by OpenAI. The hyperparameters including the number of epochs, batch size and learning rate multiplier work best for finetuning, in the applications studies herein.

In doing the 10-fold CV, all the results reported are the average of the ten folds, together with its standard deviation. The statistical significance between the models is performed via the Kruskal-Wallis H-test. The Kruskal-Wallis H test was used for sample comparison because it is non-parametric and hence does not assume that the samples are normally distributed.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A method of training a machine-learning model to detect a neurological condition, the method comprising:
providing a neurological condition dataset comprising neurological condition text embeddings, the neurological condition text embeddings including large language model (LLM) text embeddings from subjects having the neurological condition;
providing a control dataset comprising control text embeddings, the control text embeddings including LLM text embeddings from healthy subjects; and
training the machine-learning model using the neurological condition dataset and the control dataset, forming a trained machine-learning model;
wherein the trained machine-learning model is configured to detect a neurological condition.

2. The method of claim 1, wherein:
providing the neurological condition dataset comprises generating the neurological condition dataset, the generating of the neurological condition dataset including:
providing neurological condition audio recordings of speech from the subjects having the neurological condition;
converting the neurological condition audio recordings to neurological condition text transcripts; and
deriving the neurological condition text embeddings from the neurological condition text transcripts using the LLM; and
providing the control dataset comprises generating the control dataset, the generating of the control dataset including:
providing control audio recordings of speech from the healthy subjects;
converting the control audio recordings to control text transcripts; and
deriving the control text embeddings from the control text transcripts using the LLM.

3. The method of claim 2, wherein:
the converting of the neurological condition audio recordings to the neurological condition text transcripts includes inputting the neurological condition audio recordings to an automatic speech recognition model; and
the converting of the control audio recordings to the control text transcripts includes inputting the control audio recordings to an automatic speech recognition model.

4. The method of claim 1, wherein the text embeddings from subjects having the neurological condition and the text embeddings from healthy subjects capture at least one of lexical, syntactic, and semantic properties.

5. The method of claim 1, wherein the LLM includes a natural language processing (NLP) LLM.

6. The method of claim 1, wherein the LLM is fine-tuned with speech transcripts.

7. The method of claim 1, wherein the neurological condition dataset and the control dataset further comprise at least one acoustic feature.

8. The method of claim 7, wherein the at least one acoustic feature includes features related to temporal analysis, frequency analysis, different aspects of speech production, and combinations thereof.

9. The method of claim 7, wherein the at least one acoustic feature is combined using concatenation.

10. The method of claim 1, wherein detecting the neurological condition includes distinguishing between subjects having the neurological condition and healthy subjects without the neurological condition.

11. The method of claim 10, wherein the machine-learning model includes support vector classifier (SVC), logistic regression (LR), or random forest (RF).

12. The method of claim 1, wherein detecting the neurological condition includes providing a severity prediction for the neurological condition.

13. The method of claim 12, wherein the machine-learning model includes a regression model.

14. The method of claim 13, wherein the regression model includes support vector regressor (SVR), ridge regression (Ridge), or random forest regressor (RFR).

15. The method of claim 1, wherein the neurological condition is dementia or Alzheimer's disease (AD).

16. A method of detecting a neurological condition in a subject, the method comprising:
providing a test dataset from the subject, the test dataset including LLM text embeddings from the subject; and
inputting the test dataset to the trained machine-learning model according to claim 1;
wherein the trained machine-learning model outputs a detection of the neurological condition based upon the test dataset.

17. The method of claim 16, wherein providing the test dataset comprises generating the test dataset, the generating of the test dataset including:
providing audio recordings of speech from the subject;
converting the audio recordings to text transcripts; and
deriving the text embeddings from the text transcripts using the LLM.

18. The method of claim 17, wherein the converting of the audio recordings to text transcripts includes inputting the audio recordings to an automatic speech recognition model.

19. A system for detecting a neurological condition in a subject, the system including:
a processor;
a memory unit; and
a communication interface;
wherein the processor is connected to the memory unit and the communication interface; and
wherein the processor and memory are configured to implement the method of claim 16.

20. The system of claim 19, further comprising a web application configured and adapted to provide a user interface for the subject.
